(19) United States
(12) Patent Application Publication
(10) Pub. No.: US 2025/0009470 A1
(43) Pub. Date: Jan. 9, 2025

(54) SYSTEMS AND METHODS FOR GENERATING STAGES FOR ORTHODONTIC TREATMENT

(71) Applicant: SDC U.S. SmilePay SPV, New York, NY (US)

(72) Inventors: EVGENY SERGEEVICH GORBOVSKOY, Moscow (RU); ANDREY LVOVICH EMELYANENKO, Moscow (RU); SERGEY NIKOLSKIY, Nashville, TN (US)

(73) Assignee: SDC U.S. SmilePay SPV, New York, NY (US)

(21) Appl. No.: 18/710,012

(22) PCT Filed: Nov. 15, 2021
(86) PCT No.: PCT/RU2021/000504

§ 371 Date: May 14, 2024

(51) Int. Cl. A61C 7/00; G16H 30/40

(52) U.S. Cl. CPC A61C 7/002; G16H 30/40

(57) ABSTRACT

Systems and methods for generating stages for a treatment plan include receiving a first three-dimensional (3D) representation of a dentition including representations of teeth of the dentition in an initial position, determining a second 3D representation including representations of the teeth in a final position, and generating one or more stages including intermediate 3D representations of the dentition by generating a first movement vector for a first tooth of the plurality of teeth, detecting a collision between the first tooth and a second tooth based on the movement vector, generating a second movement vector for the first tooth, and generating a first stage of the one or more stages according to the second movement vector for the first tooth.

### TECHNICAL FIELD

The present disclosure relates generally to the field of dental treatment, and more specifically to systems and methods for generating a treatment plan for orthodontic care.

### BACKGROUND

Some patients may receive treatment for misalignment of teeth using dental aligners. To provide the patient with dental aligners to treat the misalignment, a treatment plan is typically generated and/or approved by a treating dentist. The treatment plan may include three-dimensional (3D) representations of the patient's teeth as they are expected to progress from their pre-treatment position (e.g., an initial position) to a target, final position selected by a treating dentist, taking into account a variety of clinical, practical and aesthetic factors. The treatment plan progression typically involves stages of treatment, including an initial stage, one or more intermediate stages, and a final stage. Each stage in the treatment plan may include a 3D representation of the patient's teeth at the corresponding stage. In developing this treatment plan, a collision may be observed to occur between two or more teeth as the teeth progress from the initial to the final position. This may require having to adjust the treatment plan, including the stages, to avoid the collision, in a final treatment plan.

### SUMMARY

In one aspect, this disclosure is directed to a method. The method includes receiving, by one or more processors, a first three-dimensional (3D) representation of a dentition including representations of a plurality of teeth of the dentition in an initial position. The method includes determining, by the one or more processors, a second 3D representation including representations of the plurality of teeth in a final position. The method further includes generating, by the one or more processors, one or more stages including intermediate 3D representations of the dentition. The intermediate 3D representations include representations of at least some of the plurality of teeth progressing from the initial position to the final position. Generating the one or more stages includes generating, by the one or more processors, a first movement vector for a first tooth of the plurality of teeth for a first intermediate 3D representation. The movement vector includes a first movement direction from the initial position for the first tooth towards the final position of the first tooth, and a first movement magnitude corresponding to a distance between the initial position and the final position. Generating the one or more stages includes detecting, by the one or more processors, a collision between the first tooth and a second tooth of the plurality of teeth based on the movement vector for the first tooth. Generating the one or more stages includes generating, by the one or more processors, a second movement vector for the first tooth for the first intermediate 3D representation. The second movement vector has at least one of a second movement direction towards the final position or a second movement magnitude. Generating the one or more stages includes generating, by the one or more processors, a first stage of the one or more stages according to the second movement vector for the first tooth.

In another aspect, this disclosure is directed to a treatment planning system. The treatment planning system includes one or more processors. The treatment planning system includes memory storing instructions that, when executed by the one or more processors, cause the one or more processors to receive a first three-dimensional (3D) representation of a dentition including representations of a plurality of teeth of the dentition in an initial position. The instructions further cause the one or more processors to determine a second 3D representation including representations of the plurality of teeth in a final position. The instructions further cause the one or more processors to generate one or more stages including intermediate 3D representations of the dentition. The intermediate 3D representations include representations of at least some of the plurality of teeth progressing from the initial position to the final position. Generating the one or more stages includes generating, by the one or more processors, a first movement vector for a first tooth of the plurality of teeth for a first intermediate 3D representation. The movement vector includes a first movement direction from the initial position for the first tooth towards the final position of the first tooth, and a first movement magnitude corresponding to a distance between the initial position and the final position. Generating the one or more stages includes detecting, by the one or more processors, a collision between the first tooth and a second tooth of the plurality of teeth based on the movement vector for the first tooth. Generating the one or more stages includes generating, by the one or more processors, a second movement vector for the first tooth for the first intermediate 3D representation. The second movement vector has at least one of a second movement direction towards the final position or a second movement magnitude. Generating the one or more stages includes generating, by the one or more processors, a first stage of the one or more stages according to the second movement vector for the first tooth.

In another aspect, this disclosure is directed to a non-transitory computer readable medium storing instructions that, when executed by one or more processors, cause the one or more processors to receive a first three-dimensional (3D) representation of a dentition including representations of a plurality of teeth of the dentition in an initial position. The instructions further cause the one or more processors to determine a second 3D representation including representations of the plurality of teeth in a final position. The instructions further cause the one or more processors to generate one or more stages including intermediate 3D representations of the dentition. The intermediate 3D representations include representations of at least some of the plurality of teeth progressing from the initial position to the final position. Generating the one or more stages includes generating, by the one or more processors, a first movement vector for a first tooth of the plurality of teeth for a first intermediate 3D representation. The movement vector includes a first movement direction from the initial position for the first tooth towards the final position of the first tooth, and a first movement magnitude corresponding to a distance between the initial position and the final position. Generating the one or more stages includes detecting, by the one or more processors, a collision between the first tooth and a second tooth of the plurality of teeth based on the movement vector for the first tooth. Generating the one or more stages includes generating, by the one or more processors, a second movement vector for the first tooth for the first intermediate 3D representation. The second movement vector has at least one of a second movement direction towards the final position or a second movement magnitude. Generating the one or more stages includes generating, by the one or more processors, a first stage of the one or more stages according to the second movement vector for the first tooth.

Various other embodiments and aspects of the disclosure will become apparent based on the drawings and detailed description of the following disclosure.

### BRIEF DESCRIPTION OF THE DRAWINGS

FIG. 1 shows a system for orthodontic treatment, according to an illustrative embodiment.

FIG. 2 shows a process flow of generating a treatment plan, according to an illustrative embodiment.

FIG. 3 shows a top-down simplified view of a model of a dentition, according to an illustrative embodiment.

FIG. 4 shows a perspective view of a three-dimensional model of the dentition of FIG. 3, according to an illustrative embodiment.

FIG. 5 shows a trace of a gingiva-tooth interface on the model shown in FIG. 3, according to an illustrative embodiment.

FIG. 6 shows selection of teeth in a tooth model generated from the model shown in FIG. 5, according to an illustrative embodiment.

FIG. 7 shows a segmented tooth model of an initial position of the dentition shown in FIG. 3, according to an illustrative embodiment.

FIG. 8 shows a target final position of the dentition from the initial position of the dentition shown in FIG. 7, according to an illustrative embodiment.

FIG. 9 shows a series of stages of the dentition from the initial position shown in FIG. 7 to the target final position shown in FIG. 8, according to an illustrative embodiment.

FIG. 10 shows a view of a dentition showing movement of a plurality of teeth from an initial position to a final position, according to an illustrative embodiment.

FIG. 11A and FIG. 11B show movement of the teeth shown in FIG. 10 in their respective stages, according to an illustrative embodiment.

FIG. 12 shows movement between two stages of a treatment plan which includes a collision between two teeth, according to an illustrative embodiment.

FIG. 13 shows initial and corrective movement vectors based on a detected collision, according to an illustrative embodiment.

FIG. 14 shows initial and corrective movement vectors based on a detected collision between a tooth and two adjacent teeth, according to an illustrative embodiment.

FIG. 15 is a flowchart showing a method of manufacturing dental aligners, according to an illustrative embodiment.

FIG. 16 is a flowchart showing a method of generating stages for a treatment plan, according to an illustrative embodiment.

FIG. 17 is a user interface showing a 3D model of a dentition, according to an illustrative embodiment.

FIG. 18 is a user interface showing the 3D model of the dentition shown in FIG. 17 following generating stages a treatment plan, according to an illustrative embodiment.

FIG. 19 is a user interface showing in the 3D model shown in FIG. 18 including a trajectory of the teeth, according to an illustrative embodiment.

FIG. 20 is a user interface showing a portion of the 3D model shown in FIG. 19 with a detailed view of the trajectory of the teeth, according to an illustrative embodiment.

### DETAILED DESCRIPTION

The present disclosure is directed to systems and methods for generating stages for orthodontic treatment. The systems and methods described herein may determine teeth movement trajectory or path form an initial position to a final position while avoiding collisions during the movement. The systems and methods described herein may determine the stages for the teeth movement trajectory according to a predefined maximum number of stages and meeting clinical limitations. The systems and methods described herein may implement different processes for determining a movement magnitude. For example, the system and methods described herein may determine the movement magnitude (or translation) according to a maximum possible velocity (or single stage movement limit) for each tooth. As another example, the systems and methods described herein may determine the movement magnitude as an average velocity (or equal movement magnitudes) for each tooth.

For each stage, the systems and methods described herein may determine a first approximation (e.g., a first movement vector) based on one of the strategies described above for determining the movement magnitude. If during a particular stage, the systems and methods described herein detect or identify a collision, the systems and methods described herein may compute or calculate an area of possible positions for each tooth constrained by clinical limitations. The area may be bounded by or defined by the 3D representation of the given tooth. The systems and methods described herein may iteratively decrease the movement magnitude (e.g., to decrease the “velocity” of the tooth) to avoid the collision. The displacement value may be proportional to an intrusion depth of one tooth to another tooth. Additionally, where the systems and methods detect or identify a collision between a tooth and two adjacent teeth, the systems and methods may compute a sum of the movement vectors (or displacement vectors). If following a movement, a particular tooth (e.g., a tooth center) is outside of an allowed area, the tooth may be iteratively moved back towards the path as needed. After each step or stage, the systems and methods described herein may iteratively determine if any collisions are detected and, if so, generate new movement vectors until the stage is collision free. For mild or moderate cases, the systems and methods described herein may converge following 20-25 iterations. On the other hand, for more complicated cases, the systems and methods described herein may converge following 100 iterations or more, for example.

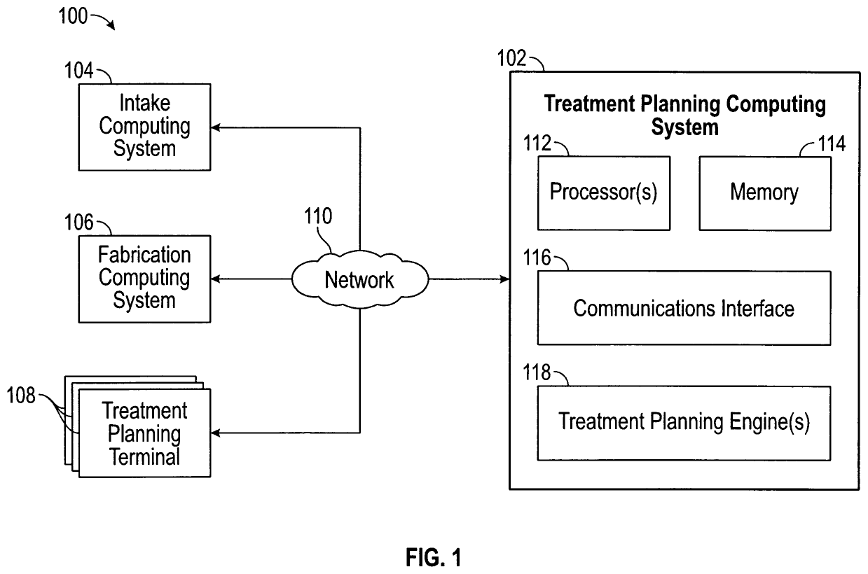

Referring to FIG. 1, a system 100 for orthodontic treatment is shown, according to an illustrative embodiment. As shown in FIG. 1, the system 100 includes a treatment plan computing system 102 communicably coupled to an intake computing system 104, a fabrication computing system 106, and one or more treatment planning terminals 108. In some embodiments, the treatment plan computing system 102 may be or may include one or more servers which are communicably coupled to a plurality of computing devices. In some embodiments, the treatment plan computing system 102 may include a plurality of servers, which may be located at a common location (e.g., a server bank) or may be distributed across a plurality of locations. The treatment plan computing system 102 may be communicably coupled to the intake computing system 104, fabrication computing system 106, and/or treatment planning terminals 108 via a communications link or network 110 (which may be or include various network connections configured to communicate, transmit, receive, or otherwise exchange data between addresses corresponding to the computing systems 102, 104, 106). The network 110 may be a Local Area Network (LAN), a Wide Area Network (WAN), a Wireless Local Area Network (WLAN), an Internet Area Network (IAN) or cloud-based network, etc. The network 110 may facilitate communication between the respective components of the system 100, as described in greater detail below.

The computing systems 102, 104, 106 include one or more processing circuits, which may include processor(s) 112 and memory 114. The processor(s) 112 may be a general purpose or specific purpose processor, an application specific integrated circuit (ASIC), one or more field programmable gate arrays (FPGAs), a group of processing components, or other suitable processing components. The processor(s) 112 may be configured to execute computer code or instructions stored in memory 114 or received from other computer readable media (e.g., CDROM, network storage, a remote server, etc.) to perform one or more of the processes described herein. The memory 114 may include one or more data storage devices (e.g., memory units, memory devices, computer-readable storage media, etc.) configured to store data, computer code, executable instructions, or other forms of computer-readable information. The memory 114 may include random access memory (RAM), read-only memory (ROM), hard drive storage, temporary storage, non-volatile memory, flash memory, optical memory, or any other suitable memory for storing software objects and/or computer instructions. The memory 114 may include database components, object code components, script components, or any other type of information structure for supporting the various activities and information structures described in the present disclosure. The memory 114 may be communicably connected to the processor 112 via the processing circuit, and may include computer code for executing (e.g., by processor(s) 112) one or more of the processes described herein.

The treatment plan computing system 102 is shown to include a communications interface 116. The communications interface 116 can be or can include components configured to transmit and/or receive data from one or more remote sources (such as the computing devices, components, systems, and/or terminals described herein). In some embodiments, each of the servers, systems, terminals, and/or computing devices may include a respective communications interface 116 which permit exchange of data between the respective components of the system 100. As such, each of the respective communications interfaces 116 may permit or otherwise enable data to be exchanged between the respective computing systems 102, 104, 106. In some implementations, communications device(s) may access the network 110 to exchange data with various other communications device(s) via cellular access, a modem, broadband, Wi-Fi, satellite access, etc. via the communications interfaces 116.

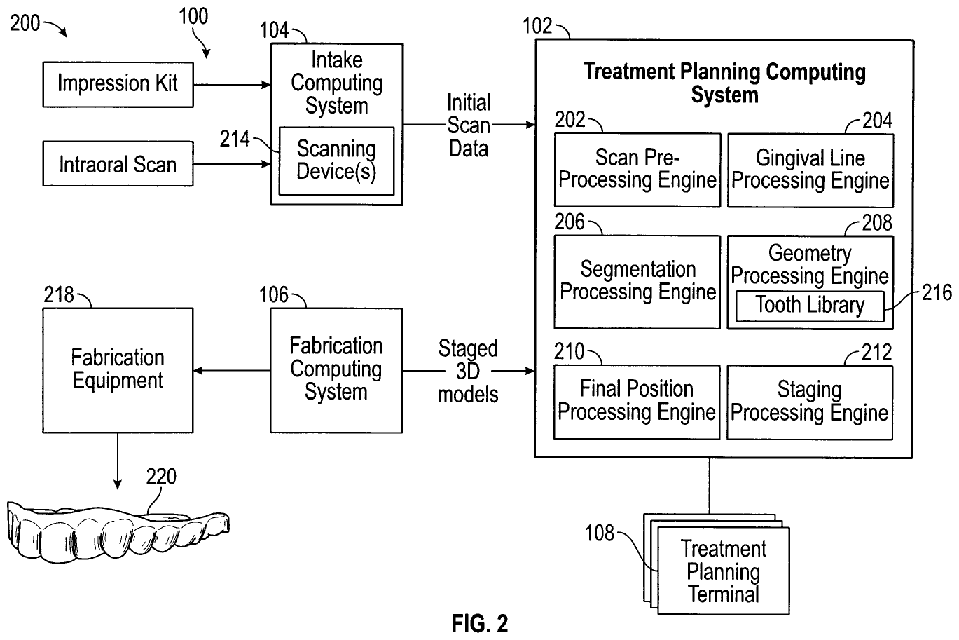

Referring now to FIG. 1 and FIG. 2, the treatment planning computing system 102 is shown to include one or more treatment planning engines 118. Specifically, FIG. 2 shows a treatment planning process flow 200 which may be implemented by the system 100 shown in FIG. 2, according to an illustrative embodiment. The treatment planning engine(s) 118 may be any device(s), component(s), circuit(s), or other combination of hardware components designed or implemented to receive inputs for and/or automatically generate a treatment plan from an initial three-dimensional (3D) model of a dentition. In some embodiments, the treatment planning engine(s) 118 may be instructions stored in memory 114 which are executable by the processor(s) 112. In some embodiments, the treatment planning engine(s) 118 may be stored at the treatment planning computing system 102 and accessible via a respective treatment planning terminal 108. As shown in FIG. 2, the treatment planning computing system 102 may include a scan pre-processing engine 202, a gingival line processing engine 204, a segmentation processing engine 206, a geometry processing engine 208, a final position processing engine 210, and a staging processing engine 212. While these engines 202-212 are shown in FIG. 2, it is noted that the system 100 may include any number of treatment planning engines 118, including additional engines which may be incorporated into, supplement, or replace one or more of the engines shown in FIG. 2.

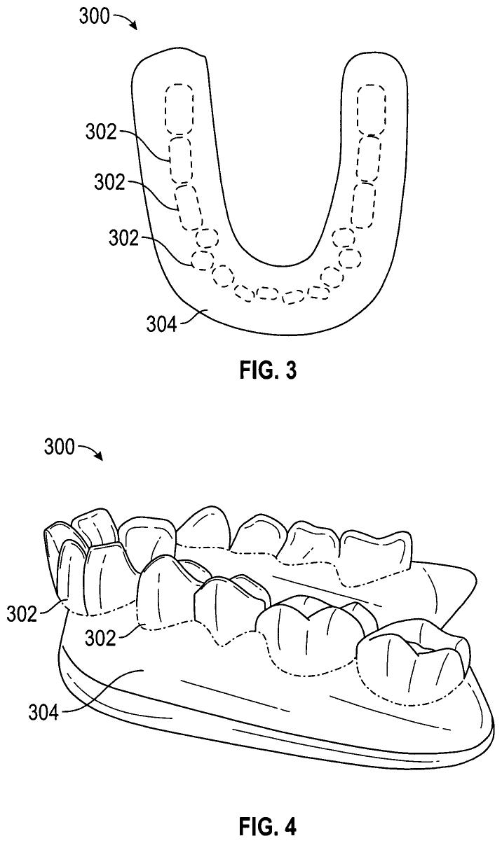

Referring to FIG. 2-FIG. 4, the intake computing system 104 may be configured to generate a 3D model of a dentition. Specifically, FIG. 3 and FIG. 4 show a simplified top-down view and a side perspective view of a 3D model of a dentition, respectively, according to illustrative embodiments. In some embodiments, the intake computing system 104 may be communicably coupled to or otherwise include one or more scanning devices 214. The intake computing system 104 may be communicably coupled to the scanning devices 214 via a wired or wireless connection. The scanning devices 214 may be or include any device, component, or hardware designed or implemented to generate, capture, or otherwise produce a 3D model 300 of an object, such as a dentition or dental arch. In some embodiments, the scanning devices 214 may include intraoral scanners configured to generate a 3D model of a dentition of a patient as the intraoral scanner passes over the dentition of the patient. For example, the intraoral scanner may be used during an intraoral scanning appointment, such as the intraoral scanning appointments described in U.S. Provisional Patent Appl. No. 62/660,141, titled “Arrangements for Intraoral Scanning,” filed Apr. 19, 2018, and U.S. patent application Ser. No. 16/130,762, titled “Arrangements for Intraoral Scanning,” filed Sep. 13, 2018. In some embodiments, the scanning devices 214 may include 3D scanners configured to scan a dental impression. The dental impression may be captured or administered by a patient using a dental impression kit similar to the dental impression kits described in U.S. Provisional Patent Appl. No. 62/522,847, titled “Dental Impression Kit and Methods Therefor,” filed Jun. 21, 2017, and U.S. patent application Ser. No. 16/047,694, titled “Dental Impression Kit and Methods Therefor,” filed Jul. 27, 2018, the contents of each of which are incorporated herein by reference in their entirety. In these and other embodiments, the scanning devices 214 may generally be configured to generate a 3D digital model of a dentition of a patient. The scanning device(s) 214 may be configured to generate a 3D digital model of the upper (i.e., maxillary) dentition and/or the lower (i.e., mandibular) dentition of the patient. The 3D digital model may include a digital representation of the patient's teeth 302 and gingiva 304. The scanning device(s) 214 may be configured to generate 3D digital models of the patient's dentition prior to treatment (i.e., with their teeth in an initial position). In some embodiments, the scanning device(s) 214 may be configured to generate the 3D digital models of the patient's dentition in real-time (e.g., as the dentition/impression) is scanned. In some embodiments, the scanning device(s) 214 may be configured to export, transmit, send, or otherwise provide data obtained during the scan to an external source which generates the 3D digital model, and transmits the 3D digital model to the intake computing system 104.

The intake computing system 104 may be configured to transmit, send, or otherwise provide the 3D digital model to the treatment planning computing system 102. In some embodiments, the intake computing system 104 may be configured to provide the 3D digital model of the patient's dentition to the treatment planning computing system 102 by uploading the 3D digital model to a patient file for the patient. The intake computing system 104 may be configured to provide the 3D digital model of the patient's upper and/or lower dentition at their initial (i.e., pre-treatment) position. The 3D digital model of the patient's upper and/or lower dentition may together form initial scan data which represents an initial position of the patient's teeth prior to treatment.

The treatment planning computing system 102 may be configured to receive the initial scan data from the intake computing system 104 (e.g., from the scanning device(s) 214 directly, indirectly via an external source following the scanning device(s) 214 providing data captured during the scan to the external source, etc.). As described in greater detail below, the treatment planning computing system 102 may include one or more treatment planning engines 118 configured or designed to generate a treatment plan based on or using the initial scan data.

Referring to FIG. 2, the treatment planning computing system 102 is shown to include a scan pre-processing engine 202. The scan pre-processing engine 202 may be or include any device(s), component(s), circuit(s), or other combination of hardware components designed or implemented to modify, correct, adjust, or otherwise process initial scan data received from the intake computing system 104 prior to generating a treatment plan. The scan pre-processing engine 202 may be configured to process the initial scan data by applying one or more surface smoothing algorithms to the 3D digital models. The scan pre-processing engine 202 may be configured to fill one or more holes or gaps in the 3D digital models. In some embodiments, the scan pre-processing engine 202 may be configured to receive inputs from a treatment planning terminal 108 to process the initial scan data. For example, the scan pre-processing engine 202 may be configured to receive inputs to smooth, refine, adjust, or otherwise process the initial scan data.

The inputs may include a selection of a smoothing processing tool presented on a user interface of the treatment planning terminal 108 showing the 3D digital model(s). As a user of the treatment planning terminal 108 selects various portions of the 3D digital model(s) using the smoothing processing tool, the scan pre-processing engine 202 may correspondingly smooth the 3D digital model at (and/or around) the selected portion. Similarly, the scan pre-processing engine 202 may be configured receive a selection of a gap filling processing tool presented on the user interface of the treatment planning terminal 108 to fill gaps in the 3D digital model(s).

In some embodiments, the scan pre-processing engine 202 may be configured to receive inputs for removing a portion of the gingiva represented in the 3D digital model of the dentition. For example, the scan pre-processing engine 202 may be configured to receive a selection (on a user interface of the treatment planning terminal 108) of a gingiva trimming tool which selectively removes gingival form the 3D digital model of the dentition. A user of the treatment planning terminal 108 may select a portion of the gingiva to remove using the gingiva trimming tool. The portion may be a lower portion of the gingiva represented in the digital model opposite the teeth. For example, where the 3D digital model shows a mandibular dentition, the portion of the gingiva removed from the 3D digital model may be the lower portion of the gingiva closest to the lower jaw. Similarly, where the 3D digital model shows a maxillary dentition, the portion of the gingiva removed from the 3D digital model may be the upper portion of the gingiva closest to the upper jaw.

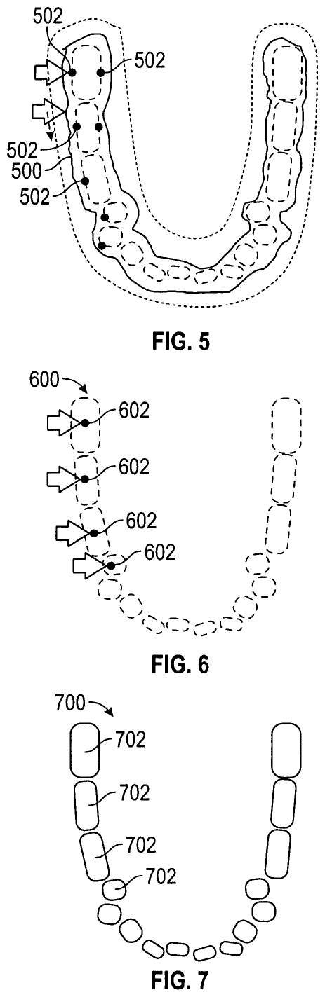

Referring now to FIG. 2 and FIG. 5, the treatment planning computing system 102 is shown to include a gingival line processing engine 204. Specifically, FIG. 5 shows a trace of a gingiva-tooth interface on the model 200 shown in FIG. 3 and FIG. 4. The gingival line processing engine 204 may be or include any device(s), component(s), circuit(s), or other combination of hardware components designed or implemented to determine, identify, or otherwise define a gingival line of the 3D digital models. The gingival line may be or include the interface between the gingiva and teeth represented in the 3D digital models. In some embodiments, the gingival line processing engine 204 may be configured to receive inputs from the treatment planning terminal 108 for defining the gingival line. The treatment planning terminal 108 may show a gingival line defining tool on a user interface which includes the 3D digital models.

The gingival line defining tool may be used for defining or otherwise determining the gingival line for the 3D digital models. As one example, the gingival line defining tool may be used to trace a rough gingival line 500. For example, a user of the treatment planning terminal 108 may select the gingival line defining tool on the user interface, and drag the gingival line defining tool along an approximate gingival line of the 3D digital model. As another example, the gingival line defining tool may be used to select (e.g., on the user interface shown on the treatment planning terminal 108) lowest points 502 at the teeth-gingiva interface for each of the teeth in the 3D digital model.

The gingival line processing engine 204 may be configured to receive the inputs provided by the user via the gingival line defining tool on the user interface of the treatment planning terminal 108 for generating or otherwise defining the gingival line. In some embodiments, the gingival line processing engine 204 may be configured to use the inputs to identify a surface transition on or near the selected inputs. For example, where the input selects a lowest point 502 (or a portion of the trace 500 near the lowest point 502) on a respective tooth, the gingival line processing engine 204 may identify a surface transition or seam at or near the lowest point 502 which is at the gingival margin. The gingival line processing engine 204 may define the transition or seam as the gingival line. The gingival line processing engine 204 may define the gingival line for each of the teeth 300 included in the 3D digital model. The gingival line processing engine 204 may be configured to generate a tooth model using the gingival line of the teeth 300 in the 3D digital model. The gingival line processing engine 204 may be configured to generate the tooth model by separating the 3D digital model along the gingival line. The tooth model may be the portion of the 3D digital model which is separated along the gingival line and includes digital representations of the patient's teeth.

Referring now to FIG. 2 and FIG. 6, the treatment planning computing system 102 is shown to include a segmentation processing engine 206. Specifically, FIG. 6 shows a view of the tooth model 600 generated by the gingival line processing engine 204. The segmentation processing engine 206 may be or include any device(s), component(s), circuit(s), or other combination of hardware components designed or implemented to determine, identify, or otherwise segment individual teeth from the tooth model. In some embodiments, the segmentation processing engine 206 may be configured to receive inputs (e.g., via a user interface shown on the treatment planning terminal 108) which select the teeth (e.g., points 602 on the teeth) in the tooth model 600. For example, the user interface may include a segmentation tool which, when selected, allows a user to select points 602 on each of the individual teeth in the tooth model 600. In some embodiments, the selection of each teeth may also assign a label to the teeth. The label may include tooth numbers (e.g., according to FDI world dental federation notation, the universal numbering system, Palmer notation, etc.) for each of the teeth in the tooth model 600. As shown in FIG. 6, the user may select individual teeth in the tooth model 600 to assign a label to the teeth.

Referring now to FIG. 7, depicted is a segmented tooth model 700 generated from the tooth model 600 shown in FIG. 6. The segmentation processing engine 206 may be configured to receive the selection of the teeth from the user via the user interface of the treatment planning terminal 108. The segmentation processing engine 206 may be configured to separate each of the teeth selected by the user on the user interface. For example, the segmentation processing engine 206 may be configured to identify or determine a gap between two adjacent points 602. The segmentation processing engine 206 may be configured to use the gap as a boundary defining or separating two teeth. The segmentation processing engine 206 may be configured to define boundaries for each of the teeth in the tooth model 600. The segmentation processing engine 206 may be configured to generate the segmented tooth model 700 including segmented teeth 702 using the defined boundaries generated from the selection of the points 602 on the teeth in the tooth model 600.

The treatment planning computing system 102 is shown to include a geometry processing engine 208. The geometry processing engine 208 may be or include any device(s), component(s), circuit(s), or other combination of hardware components designed or implemented to determine, identify, or otherwise generate whole tooth models for each of the teeth in the 3D digital model. Once the segmentation processing engine 206 generates the segmented tooth model 700, the geometry processing engine 208 may be configured to use the segmented teeth to generate a whole tooth model for each of the segmented teeth. Since the teeth have been separated along the gingival line by the gingival line processing engine 204 (as described above with reference to FIG. 6), the segmented teeth may only include crowns (e.g., the segmented teeth may not include any roots). The gingival line processing engine 204 may be configured to generate a whole tooth model including both crown and roots using the segmented teeth. In some embodiments, the segmentation processing engine 206 may be configured to generate the whole tooth models using the labels assigned to each of the teeth in the segmented tooth model 700. For example, the geometry processing engine 208 may be configured to access a tooth library 216. The tooth library 216 may include a library or database having a plurality of whole tooth models. The plurality of whole tooth models may include tooth models for each of the types of teeth in a dentition. The plurality of whole tooth models may be labeled or grouped according to tooth numbers.

The geometry processing engine 208 may be configured to generate the whole tooth models for a segmented tooth by performing a look-up function in the tooth library 216 using the label assigned to the segmented tooth to identify a corresponding whole tooth model. The geometry processing engine 208 may be configured to morph the whole tooth model identified in the tooth library 216 to correspond to the shape (e.g., surface contours) of the segmented tooth. In some embodiments, the geometry processing engine 208 may be configured to generate the whole tooth model by stitching the morphed whole tooth model from the tooth library 216 to the segmented tooth, such that the whole tooth model includes a portion (e.g., a root portion) from the tooth library 216 and a portion (e.g., a crown portion) from the segmented tooth. In some embodiments, the geometry processing engine 208 may be configured to generate the whole tooth model by replacing the segmented tooth with the morphed tooth model from the tooth library. In these and other embodiments, the geometry processing engine 208 may be configured to generate whole tooth models, including both crown and roots, for each of the teeth in a 3D digital model. The whole tooth models of each of the teeth in the 3D digital model may depict, show, or otherwise represent an initial position of the patient's dentition.

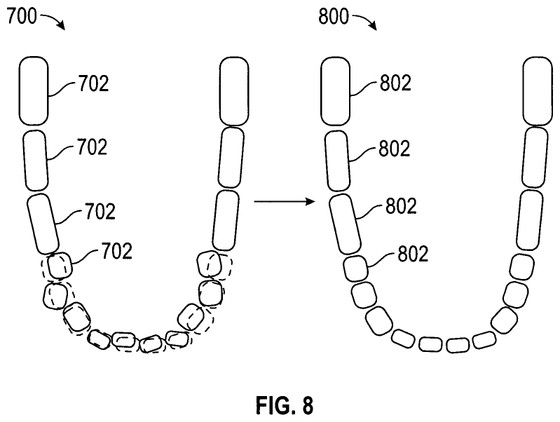

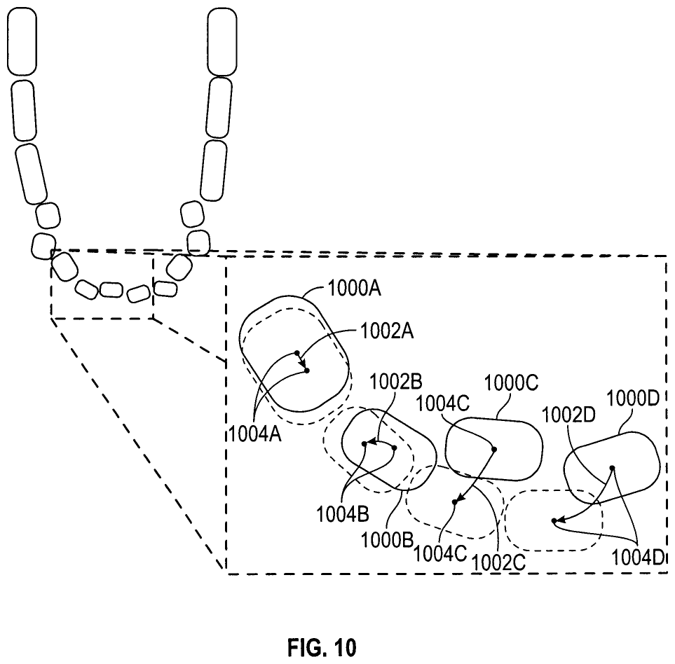

Referring now to FIG. 2, FIG. 8, and FIG. 10, the treatment planning computing system 102 is shown to include a final position processing engine 210. FIG. 8 shows one example of a target final position of the dentition from the initial position of the dentition shown in FIG. 7 from a top-down view. FIG. 10 shows one example of a target final position of the dentition from the initial position of the dentition shown in FIG. 7 from a side view. Specifically, FIG. 10 shows one example of a target final position of each of the upper and lower detentions relative to an occlusal axis, such as the longitudinal axis of each tooth (e.g., the axis extending between the upper and lower dentition), as will be described below.

The final position processing engine 210 may be or may include any device(s), component(s), circuit(s), or other combination of hardware components designed or implemented to determine, identify, or otherwise generate a final position of the patient's teeth. The final position processing engine 210 may be configured to generate the treatment plan by manipulating individual 3D models of teeth within the 3D model (e.g., shown in FIG. 7). In some embodiments, the final position processing engine 210 may be configured to receive inputs for generating the final position of the patient's teeth. The final position may be a target position of the teeth post-orthodontic treatment or at a last stage of realignment. A user of the treatment planning terminal 108 may provide one or more inputs for each tooth or a subset of the teeth in the initial 3D model to move the teeth from their initial position to their final position (shown in dot-dash). For example, the treatment planning terminal 108 may be configured to receive inputs to drag, shift, rotate, or otherwise move individual teeth to their final position, incrementally shift the teeth to their final position, etc. The movements may include lateral/longitudinal movements, rotational movements, translational movements, etc. The movements may include intrusions and/or extrusions of the teeth relative to the occlusal axis, as will be described below.

In some embodiments, the manipulation of the 3D model may show a final (or target) position of the teeth of the patient following orthodontic treatment or at a last stage of realignment via dental aligners. In some embodiments, the final position processing engine 210 may be configured to apply one or more movement thresholds (e.g., a maximum lateral and/or rotational movement for treatment) to each of the individual 3D teeth models for generating the final position. As such, the final position may be generated in accordance with the movement thresholds.

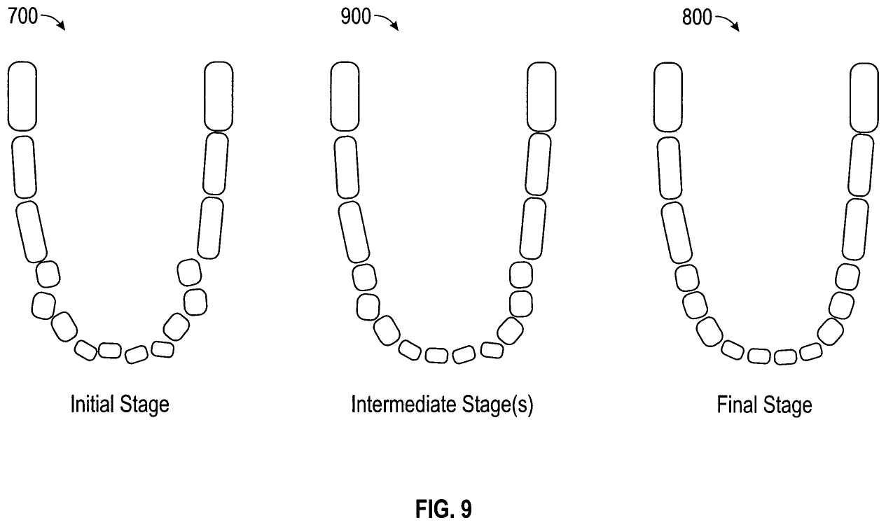

Referring now to FIG. 2 and FIG. 9, the treatment planning computing system 102 is shown to include a staging processing engine 212. Specifically, FIG. 9 shows a series of stages of the dentition from the initial position shown in FIG. 7 to the target final position shown in FIG. 8 and FIG. 10, according to an illustrative embodiment. The staging processing engine 212 may be or include any device(s), component(s), circuit(s), or other combination of hardware components designed or implemented to determine, identify, or otherwise generate stages of treatment from the initial position to the final position of the patient's teeth. In some embodiments, the staging processing engine 212 may be configured to receive inputs (e.g., via a user interface of the treatment planning terminal 108) for generating the stages. In some embodiments, the staging processing engine 212 may be configured to automatically compute or determine the stages based on the movements from the initial to the final position. The staging processing engine 212 may be configured to apply one or more movement thresholds (e.g., a maximum lateral and/or rotational movement for a respective stage) to each stage of treatment plan. The staging processing engine 212 may be configured to generate the stages as 3D digital models of the patient's teeth as they progress from their initial position to their final position. For example, and as shown in FIG. 9, the stages may include an initial stage including a 3D digital model of the patient's teeth at their initial position, one or more intermediate stages including 3D digital model(s) of the patient's teeth at one or more intermediate positions, and a final stage including a 3D digital model of the patient's teeth at the final position.

In some embodiments, the staging processing engine 212 may be configured to generate at least one intermediate stage for each tooth based on a difference between the initial position of the tooth and the final position of the tooth. For instance, where the staging processing engine 212 generates one intermediate stage, the intermediate stage may be a halfway point between the initial position of the tooth and the final position of the tooth. Each of the stages may together form a treatment plan for the patient, and may include a series or set of 3D digital models.

Following generating the stages, the treatment planning computing system 102 may be configured to transmit, send, or otherwise provide the staged 3D digital models to the fabrication computing system 106. In some embodiments, the treatment planning computing system 102 may be configured to provide the staged 3D digital models to the fabrication computing system 106 by uploading the staged 3D digital models to a patient file which is accessible via the fabrication computing system 106. In some embodiments, the treatment planning computing system 102 may be configured to provide the staged 3D digital models to the fabrication system 106 by sending the staged 3D digital models to an address (e.g., an email address, IP address, etc.) for the fabrication computing system 106.

The fabrication computing system 106 can include a fabrication computing device and fabrication equipment 218 configured to produce, manufacture, or otherwise fabricate dental aligners. The fabrication computing system 106 may be configured to receive a plurality of staged 3D digital models corresponding to the treatment plan for the patient. As stated above, each 3D digital model may be representative of a particular stage of the treatment plan (e.g., a first 3D model corresponding to an initial stage of the treatment plan, one or more intermediate 3D models corresponding to intermediate stages of the treatment plan, and a final 3D model corresponding to a final stage of the treatment plan).

The fabrication computing system 106 may be configured to send the staged 3D models to fabrication equipment 218 for generating, constructing, building, or otherwise producing dental aligners 220. In some embodiments, the fabrication equipment 218 may include a 3D printing system. The 3D printing system may be used to 3D print physical models corresponding the 3D models of the treatment plan. As such, the 3D printing system may be configured to fabricate physical models which represent each stage of the treatment plan. In some implementations, the fabrication equipment 218 may include casting equipment configured to cast, etch, or otherwise generate physical models based on the 3D models of the treatment plan. Where the 3D printing system generates physical models, the fabrication equipment 218 may also include a thermoforming system. The thermoforming system may be configured to thermoform a polymeric material to the physical models, and cut, trim, or otherwise remove excess polymeric material from the physical models to fabricate a dental aligner. In some embodiments, the 3D printing system may be configured to directly fabricate dental aligners 220 (e.g., by 3D printing the dental aligners 220 directly based on the 3D models of the treatment plan). Additional details corresponding to fabricating dental aligners 220 are described in U.S. Provisional Patent Appl. No. 62/522,847, titled “Dental Impression Kit and Methods Therefor,” filed Jun. 21, 2017, and U.S. patent application Ser. No. 16/047,694, titled “Dental Impression Kit and Methods Therefor,” filed Jul. 27, 2018, and U.S. Pat. No. 10,315,353, titled “Systems and Methods for Thermoforming Dental Aligners,” filed Nov. 13, 2018, the contents of each of which are incorporated herein by reference in their entirety.

The fabrication equipment 218 may be configured to generate or otherwise fabricate dental aligners 220 for each stage of the treatment plan. In some instances, each stage may include a plurality of dental aligners 220 (e.g., a plurality of dental aligners 220 for the first stage of the treatment plan, a plurality of dental aligners 220 for the intermediate stage(s) of the treatment plan, a plurality of dental aligners 220 for the final stage of the treatment plan, etc.). Each of the dental aligners 220 may be worn by the patient in a particular sequence for a predetermined duration (e.g., two weeks for a first dental aligner 220 of the first stage, one week for a second dental aligner 220 of the first stage, etc.).

Referring now to FIG. 10, depicted is a view of a dentition showing movement of a plurality of teeth from an initial position to a final position, according to an illustrative embodiment. As described above, the final position for each tooth 1000 (shown in dashed line) may be defined, determined, or otherwise generated by the final position processing engine 210. As described above and in greater detail below, the staging processing engine 212 may be configured to generate one or more stages which includes representations of the teeth 1000 progressing from the initial position (shown in solid line) to the final position.

The staging processing engine 212 may be configured to determine movement distances for each tooth 1000 in the dentition. As described above with reference to FIG. 8, during orthodontic treatment via dental aligners, various teeth 1000 within a dentition may be moved at least some distance from an initial position to a final position. The staging processing engine 212 may be configured to determine movement distances for each of the teeth 1000 which are moved from an initial position to a final position. The staging processing engine 212 may be configured to determine the movement distance based on a path 1002 for each tooth 1000 from the initial position to the final position. The path 1002 for each tooth 1000, as shown in FIG. 10, may show a movement of a center 1004 of the tooth 1000 from the initial position to the final position. Some teeth 1000 (such as tooth 1000A) may have relatively straight paths 1002 such that the path 1002 includes translational movements. Other teeth 1000 (such as teeth 1000B-1000D) may have curved paths 1002 such that the path 1002 includes both rotational and translational movements.

In some embodiments, the staging processing engine 212 may be configured to determine the movement distance as the total length of the path 1002 (e.g., a curved distance as opposed to a straight line distance). In some embodiments, the staging processing engine 212 may be configured to determine the movement distance as a straight line distance of the center 1004 of a tooth 1000 from the initial position to the final position. As shown in FIG. 10, some teeth 1000 may have a greater movement distance than other teeth 1000. For instance, in the example dentition shown in FIG. 10, of each of the teeth 1000A-1000D, tooth 1000A may have the shortest movement distance, whereas tooth 1000D may have the longest movement distance. The staging processing engine 212 may be configured to use the movement distances for each of the teeth 1000 to determine a number of stages to generate.

The staging processing engine 212 may be configured to determine or identify a maximum movement distance. In some embodiments, the staging processing engine 212 may be configured to identify a maximum movement distance for a respective tooth 1000 of the plurality of teeth 1000 from the initial position to the final position. The staging processing engine 212 may be configured to compare the movement distances determined for each tooth 1000 to identify the maximum movement distance. The staging processing engine 212 may be configured to use the maximum movement distance to determine a number of stages which are to be generated for a patient. In other words, the number of stages for a patient may be a function of the maximum movement distance for a tooth of the patient. As the maximum movement distance for the patient decreases, the number of stages for the treatment plan may correspondingly decrease.

The staging processing engine 212 may be configured to maintain, include, retrieve, or otherwise identify a single stage movement limit. The single stage movement limit may be a limit of a distance a tooth may be moved in one stage of the treatment plan. In some embodiments, the single stage movement limit may be 1.0 mm. In some embodiments, the single stage movement may be less than or greater than 1.0 mm, such as 0.5 mm, 0.6 mm, 0.7 mm, 0.8 mm, 0.9 mm, 1.1 mm, 1.2 mm, 1.3 mm, 1.4 mm, 1.5 mm, etc. The staging processing engine 212 may be configured to apply the single stage movement limit to the maximum movement distance to determine a number of stages to generate for the treatment plan. In some embodiments, the staging processing engine 212 may be configured to determine the number of stages by dividing the maximum movement distance by the single stage movement limit. For example, where the maximum movement distance is 7.0 mm and the single stage movement limit is 1.0 mm, the staging processing engine 212 may determine to generate seven stages for the patient. Additionally, where the maximum movement distance is not equally divided by the single stage movement limit, the staging processing engine 212 may be configured to round up the determined number of stages. For example, where the maximum movement distance is 4.9 mm and the single stage movement limit is 1.0 mm, the staging processing engine 212 may determine to generate five stages for the patient.

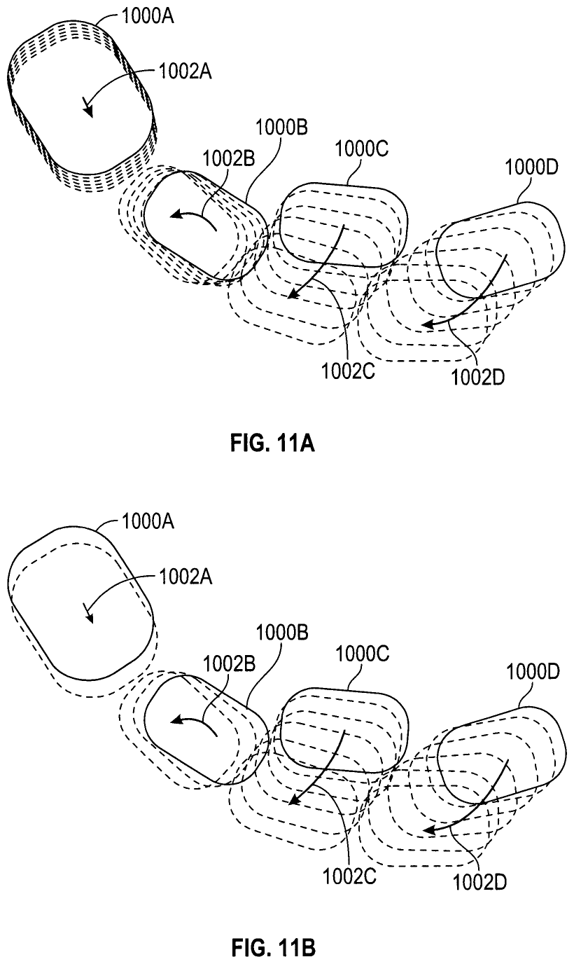

Referring now to FIG. 11A and FIG. 11B, the staging processing engine 212 may be configured to generate one or more stages which includes intermediate 3D representations of the dentition. Specifically, FIG. 11A and FIG. 11B shows movement of the teeth 1000A-1000D shown in FIG. 10 in their respective stages, according to an illustrative embodiment. The staging processing engine 212 may be configured to generate stages for a treatment plan to show movement of the teeth 1000 progressing from the initial position to the final position. In the example shown in FIG. 11, the staging processing engine 212 may generate five stages for the patient. The staging processing engine 212 may be configured to determine, compute, or otherwise generate a movement vector for each tooth 1000 which is to be moved for a given stage. In some embodiments, the staging processing engine 212 may be configured to generate the movement vector as a function of a current position of the tooth 1000 (at a given stage) and a target position of the tooth 1000 (at a subsequent stage). As such, for each stage in which a tooth 1000 is moved, the movement vector across the stages may generally follow the path 1002 of the tooth 1000 from the initial position of the tooth to the final position of the tooth. Where the path 1002 of the tooth 1000 is a relatively straight path 1002 (such as for tooth 1000A), the movement vectors along the path 1002 for the tooth 1000 may generally include translational movements. On the other hand, where the path 1002 of a tooth 1000 is a curved path 1002 (such as for teeth 1000B-1000D), the movement vector along the path 1002 for the tooth 1000 may generally include translational and rotational movements. The movement vectors may include a direction component and a magnitude component (or a direction and a magnitude). The direction component may define the translational movement of the tooth 1000 along the path. The magnitude component may define a length the tooth 1000 is moved in the direction.

In some embodiments, teeth 1000 which are to be moved from an initial position to a final position may be moved at each stage of the treatment plan. Specifically, FIG. 11A shows stages of a treatment plan which includes movement of each of the teeth 1000A-1000D at each stage of the treatment plan. The staging processing engine 212 may be configured to generate movement vectors for each of the teeth 1000A-1000D for each stage of the treatment plan. In some embodiments, the staging processing engine 212 may be configured to generate the movement vectors for a respective tooth 1000 as a function of the path 1002 for the tooth and the number of stages. For instance, the staging processing engine 212 may be configured to generate the movement vectors by dividing the path 1002 into equal segments. In other words, the staging processing engine 212 may generate a segmented path from the path 1002. In the example shown in FIG. 11A, each segment may have a magnitude (or magnitude component) which corresponds to the number of stages. As one example, where the path 1002 has a movement distance equal to 4.0 mm and the staging processing engine 212 is to generate four stages, the staging processing engine 212 may generate four movement vectors, where each movement vector has a respective direction component which follows the path 1002 and a magnitude component equal to 1.0 mm. By providing more incremental or granular movements of the teeth at each stage of the treatment plan, the teeth may be less likely to revert back to a prior or previous position. Such implementations and embodiments may provide for more successful treatment plans (e.g., treatment outcomes that are more consistent with the target final position and are less likely to have reversions back to a prior position inconsistent with the target final position).

In some embodiments, teeth 1000 which are to be moved from an initial position to a final position may be moved in a subset of stages of the treatment plan. Specifically, FIG. 11B shows stages of a treatment plan which includes movement of some, but not all, of the teeth 1000A-1000D at each stage of the treatment plan. The staging processing engine 212 may be configured to generate movement vectors for each of the teeth 1000A-1000D for the initial stage(s) of the treatment plan until teeth 1000A-1000D are in their final position. In some embodiments, the staging processing engine 212 may be configured to generate the movement vectors for a respective tooth 1000 as a function of the path 1002 for the tooth and the single stage movement limit. In some embodiments, the staging processing engine 212 may be configured to generate first movement vectors for respective teeth 1000 at the first stage of the treatment plan starting from the initial position of the tooth 1000, with the movement vector having a direction component along the path 1002 and a magnitude component equal to the single stage movement limit (or less than the single stage movement limit, if the path 1002 has a movement distance less than the single stage movement limit). The staging processing engine 212 may be configured to generate second and subsequent movement vectors (as needed) for the respective teeth 1000 until the teeth 1000 are in their final position.

As shown in the example depicted in FIG. 11B, the first tooth 1000A may be moved at the first stage, the second tooth 1000B may be moved at the first stage and the second stage, and the third and fourth tooth 1000C, 1000D may be moved at the first stage through the fifth stage. In this example, and assuming a single stage movement limit of 1.00 mm, the path 1002A for the first tooth 1000A may be less than or equal to 1.00 mm, the path 1002B for the second tooth 1000B may be between 1.01 mm and 2.00 mm, and the paths 1002C-1002D may be between 4.01 mm and 5.00 mm. Additionally, in a final movement stage for a respective tooth 1000, the tooth 1000 may move less than or equal to the single stage movement limit (e.g., 1.00 mm). The staging processing engine 212 may be configured to generate the movement vector for the final movement stage by, for example, subtracting the movement distance from the sum of the magnitude of the movement vectors from the previous stage(s) (e.g., the number of stages multiplied by the single stage movement limit).

Following generating the movement vectors for each of the teeth 1000 as described above with reference to FIG. 11A and FIG. 11B, the staging processing engine 212 may be configured to project each of the teeth 1000 according to respective movement vectors along the path 1002. Specifically, since each tooth 1000 is represented as a three-dimensional object, the staging processing engine 212 may be configured to project, transpose, or otherwise move the 3D digital representation of the tooth 1000 using the respective movement vector. The staging processing engine 212 may be configured to project the 3D representation of the tooth 1000 while maintaining the 3D structure of the tooth 1000. In other words, the staging processing engine 212 may be configured to perform an affine transformation of the tooth 1000 in accordance with the movement vector.

The staging processing engine 212 may be configured to project each tooth 1000 according to the movement vectors for each stage of the treatment plan. The staging processing engine 212 may be configured to project a respective tooth 1000 for a stage from a current position (e.g., a position of the tooth 1000 at a current stage) in a direction according to the direction component of the movement vector, and a distance or magnitude in the direction according to the magnitude component of the movement vector.

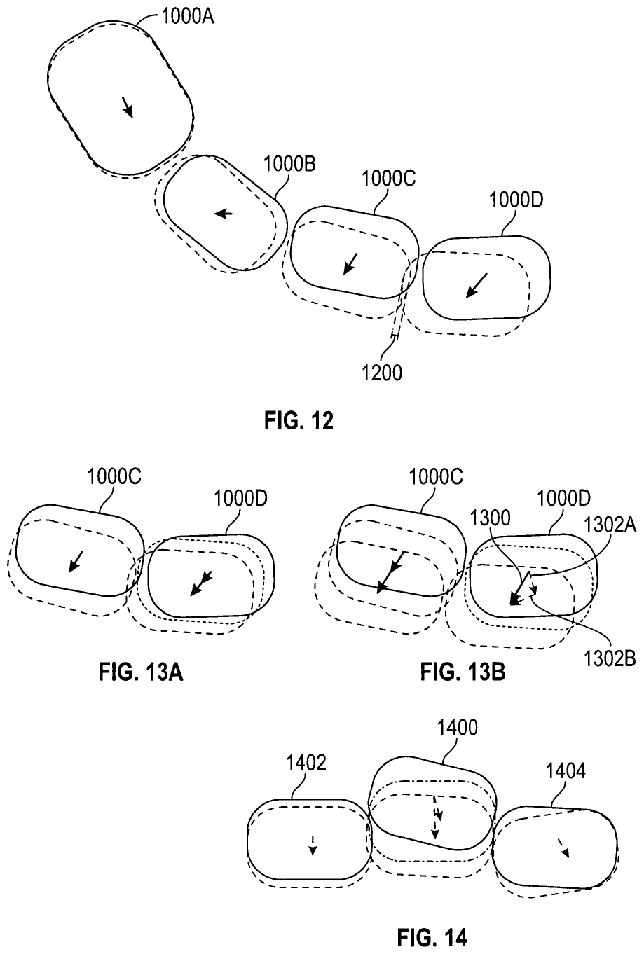

Referring now to FIG. 12, at each stage, following projecting each tooth 1000, the staging processing engine 212 may be configured to identify any collisions between two or more teeth 1000. Specifically, FIG. 12 shows movement between two stages of a treatment plan which includes a collision between two teeth, according to an illustrative embodiment. The staging processing engine 212 may be configured to identify or detect collisions based on two or more teeth 1000 at least partially overlapping, intersecting, or otherwise intruding one another. Since teeth are rigid 3D structures, during a collision, teeth will either not move any further or will break. As such, where the staging processing engine 212 detects a collision between two or more teeth 1000, the staging processing engine 212 may be configured to determine or generate corrective movement vectors for one or more of the colliding teeth 1000. In the example shown in FIG. 12, the third and fourth teeth 1000C, 1000D are shown to have a collision between one another as the teeth 1000C, 1000D progress between their respective current position (shown in solid) and subsequent position (shown in dash).

In some embodiments, the staging processing engine 212 may be configured to detect, compute, or otherwise determine an intrusion depth 1200 of the collision between the teeth. The intrusion depth 1200 refers to a depth, degree, or distance in which the teeth overlap in the collision. The staging processing engine 212 may be configured to compute or otherwise determine the intrusion depth 1200 by identifying the outermost points on the two colliding teeth (e.g., a point on a respective tooth which overlaps the adjacent, colliding tooth to a greatest degree, or is located closest to a center of the adjacent, colliding tooth). The staging processing engine 212 may be configured to compute or otherwise determine the intrusion depth 1200 as the distance between the outermost points on the two colliding teeth.

Referring now to FIG. 12 and FIG. 13A through FIG. 1313, the staging processing engine 212 may be configured to determine or generate corrective movement vectors to avoid the collision between the colliding teeth. Specifically, FIG. 13A and FIG. 13B show example initial and corrective movement vectors based on a detected collision, according to an illustrative embodiment. In some embodiments, the staging processing engine 212 may be configured to generate the corrective movement vectors by modifying the magnitude component of the initial movement vector. For example, the staging processing engine 212 may be configured to modify the magnitude component of the initial movement vector proportionally to the determined intrusion depth 1200.

In some embodiments, and as shown in FIG. 13A, the staging processing engine 212 may be configured to modify the magnitude component of the initial movement vector, but maintain the direction component of the initial movement vector, for generating the corrective movement vector. The staging processing engine 212 may be configured to generate the corrective movement vector such that the tooth 1000 continues to move along the same path 1002, but at a “slower” pace (e.g., a having a smaller or shorter magnitude component but in the same direction). In the example shown in FIG. 13A, where the tooth 1000D is projected from the current position (shown in solid) according to the initial movement vector to the target position (shown in dash), the tooth 1000D collides with the adjacent tooth 1000C. On the other hand, where the tooth 1000D is projected from the current position according to the corrective movement vector (shown in dot), the tooth 100D does not collide with the adjacent tooth 1000C.

In some embodiments, and as shown in FIG. 13B, the staging processing engine 212 may be configured to modify the direction component of the initial movement vector, but maintain the magnitude component of the initial movement vector, as part of generating the corrective movement vector. For example, the initial movement vector may have a direction component which is on the path 1300 from the initial position for the tooth 1000D, which would result in a collision with the adjacent tooth 1000C. The staging processing engine 212 may be configured to modify the direction component to be adjacent to the path 1300 but still towards the final position of the tooth. For example, the direction component for the corrective movement vector and the direction component for the initial movement vector (and/or the path 1300) may form an angle between approximately 1° and 90°. In some embodiments, the direction component for the corrective movement vector may form an acute angle between the direction components.

In some embodiments, the staging processing engine 212 may be configured to iteratively modify the direction component to be adjacent to the path 1300 according to the intrusion depth 1200. In some embodiments, the staging processing engine 212 may be configured to apply one or more tolerances or thresholds to the modified direction component. For example, the tolerance may be a distance or measurement of a deviation from the path 1300. The staging processing engine 212 may be configured to iteratively modify the direction component up to the tolerance of the deviation. Once the direction component meets the tolerance, the staging processing engine 212 may be configured to modify the movement vector for other teeth to avoid the collision.

As shown in FIG. 13B, where the staging processing engine 212 generates a corrective movement vector 1302A which is adjacent to the path 1300, the staging processing engine 212 may generate a movement vector 1302B which brings the tooth 1000D back on the path 1300. The movement vector 1302B may bring the tooth 1000D back on the path 1300 and at a position which is different from a previous position of the tooth 1000D (e.g., different from the position prior to the stage associated with the corrective movement vector 1302A). In this regard, the movement vectors 1302A, 1302B may not result in round-tripping the tooth 1000D, which typically involves moving the tooth from a first position to a second position and back to the first position to avoid a collision. Instead, the tooth 1000D is still moved, advanced, or otherwise progressed along the path 1300 to different positions, while still avoiding a collision with the adjacent tooth 1000C. As shown in FIG. 13B, the staging processing engine may generate the movement vector 1302B which includes a third movement direction and magnitude. In some instances, the magnitude of the movement vector 1302B may be the same as the magnitude of the corrective movement vector 1302A. In other instances, the magnitude of the movement vector 1302B may be different (e.g., less than or greater than) the magnitude of the corrective movement vector 1302A. The third movement direction may be towards the final position of the first tooth. In this regard, the tooth 1000D may be moved towards the final position, rather than back to a previous position as is typically performed during round-tripping. The third movement direction may cause the tooth 1000D to move from adjacent to the path 1300 (e.g., at a second position) to on the path 1300 (e.g., at a third position). In some embodiments, each movement direction may move the tooth 1000D closer to the final position. In this regard, a position of the tooth prior to movement at the stage corresponding to the first movement vector 1302A may be further from the final position than the stage corresponding to the movement vector 1302B. In other words, the second position (e.g., following the stage corresponding to the first movement vector 1302A) is closer to the final position than the first position (prior to the stage corresponding to the movement vector 1302A), and the third position (e.g., following the stage corresponding to the second movement vector 13028) is closer to the final position than the second position.

In some embodiments, the movement vector 1302B may be associated with the next stage (e.g., immediately subsequent stage) following the stage associated with the corrective movement vector 1302A. In some embodiments, the movement vector 1302B may be associated with any subsequent stage following the stage associated with the corrective movement vector 1302A.

Referring now to FIG. 14, and similar to detecting collisions between two teeth, in some instances, the staging processing engine 212 may identify or detect collisions between a tooth and two adjacent teeth. Specifically, FIG. 14 shows initial and corrective movement vectors based on a detected collision between a tooth 1400 and two adjacent teeth 1402, 1404, according to an illustrative embodiment. In instances in which the staging processing engine 212 detects a collision between a tooth 1400 and two adjacent teeth 1402, 1404, the staging processing engine 212 may be configured to determine or otherwise generate a corrective movement vector as a function of the movement vectors for the adjacent teeth 1402, 1404. For example, the staging processing engine 212 may be configured to determine the direction component of the corrective movement vector as a sum of the movement vectors for the adjacent teeth 1402, 1404. Additionally, the staging processing engine 212 may be configured to determine the magnitude component of the corrective movement vector as the sum of the movement vectors. The staging processing engine 212 may be configured to iteratively decrease the magnitude component of the corrective movement vector (or modify the direction component of the corrective movement vector) until the staging processing engine 212 no longer detects collisions with the adjacent teeth 1402, 1404. In the example shown in FIG. 14, where the tooth 1400 is projected from the current position (shown in solid) according to the initial movement vector to the target position (shown in dash), the tooth 1400 collides with both the adjacent teeth 1402, 1404. On the other hand, where the tooth 1400 is projected from the current position according to the corrective movement vector (shown in dot-dash), the tooth 1400 does not collide with the adjacent teeth 1402, 1404.

The staging processing engine 212 may be configured to iteratively evaluate the projected positions of the teeth at each stage according to movement vectors (initial and corrective) until the staging processing engine 212 does not detect any collisions at any stages. Following the staging processing engine 212 generating initial and corrective (as needed) movement vectors which do not result in any collisions, the staging processing engine 212 may be configured to transmit, send, or otherwise provide the staged 3D models to the fabrication computing system 106 as described in greater detail above.

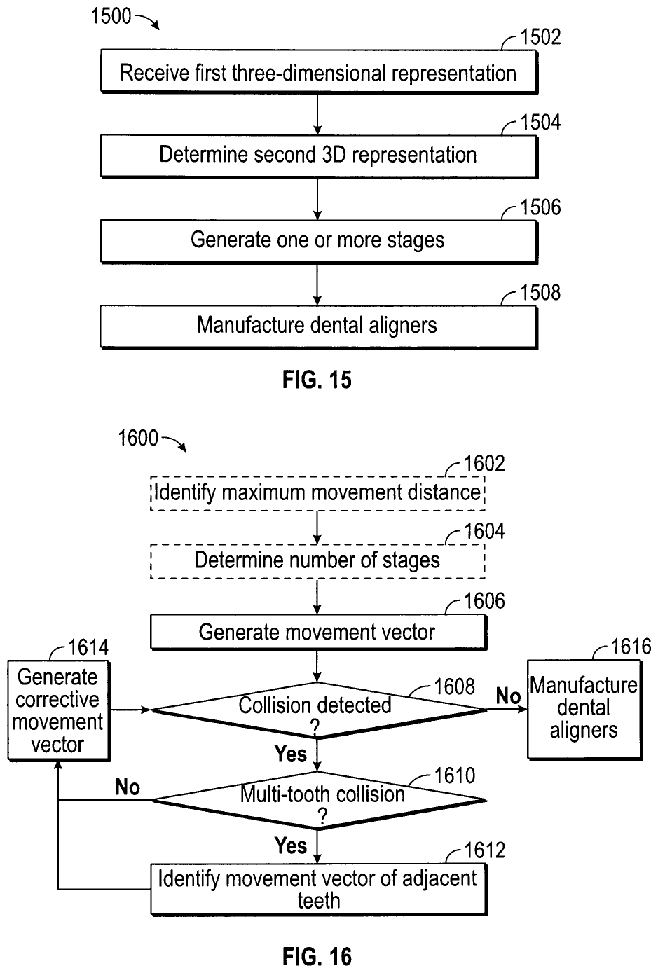

Referring now to FIG. 15, depicted is a flowchart showing a method 1500 of manufacturing dental aligners, according to an illustrative embodiment. The steps of the method 1500 may be performed by one or more of the components described above with reference to FIG. 1-FIG. 14.

At step 1502, the final position processing engine 210 may receive a first 3D representation. In some embodiments, the final position processing engine 210 may receive a first 3D representation of a dentition including representations of a plurality of teeth of the dentition in an initial position. In some embodiments, the final position processing engine 210 may receive the first 3D representation from the scanning devices 214 described above with reference to FIG. 2. For example, the final position processing engine 210 may receive the first 3D representation from a scanning device 214 which scanned a patient's dentition (e.g., directly as an intraoral scanner, or indirectly by scanning impressions captured by the patient). In some embodiments, the final position processing engine 210 may receive the initial 3D representation from one of the engines of the treatment planning computing system 102 (such as the geometry processing engine 208, for example).

At step 1504, the final position processing engine 210 may determine a second 3D representation. In some embodiments, the final position processing engine 210 may determine a second 3D representation including representations of the plurality of teeth in a final position. The final position processing engine 210 may determine the second 3D representation as described above with reference to FIG. 8. In some embodiments, the final position processing engine 210 may automatically determine the second 3D representation (e.g., without any user input or feedback). In some embodiments, the final position processing engine 210 may determine the second 3D representations based on inputs received from the treatment planning terminal 108.

At step 1506, the staging processing engine 212 may generate one or more stages. In some embodiments, the staging processing engine 212 may generate one or more stages including intermediate 3D representations of the dentition. The intermediate 3D representations may include representations of at least some of the plurality of teeth progressing from the initial position to the final position. Additional details regarding step 1506 are described below with reference to FIG. 16.

At step 1508, the fabrication equipment 218 may manufacture a plurality of dental aligners 220. In some embodiments, the fabrication equipment 218 may manufacture a plurality of aligners configured to move the at least some teeth from the initial position to the final position for each stage of the one or more stages. In some embodiments, the fabrication equipment 218 may manufacture the aligners by 3D printing physical models from the initial, intermediate, and final 3D representations of the dentition. The fabrication equipment 218 may then manufacture the aligners by thermoforming aligner material to the physical models. In some embodiments, the fabrication equipment 218 may manufacture the aligners by 3D printing the aligners from the initial, intermediate, and final 3D representations of the dentition.

Referring now to FIG. 16, depicted is a flowchart showing a method 1600 of generating stages for a treatment plan, according to an illustrative embodiment. The steps of the method 1600 may be performed by one or more of the components described above with reference to FIG. 1-FIG. 14. Additionally, and as described above, step 1506 of FIG. 15 may include each or a subset of the steps in method 1600 and shown in FIG. 16.

At step 1602, and optionally, the staging processing engine 212 identifies a maximum movement distance. In some embodiments, the staging processing engine 212 may identify, for the plurality of teeth, a maximum movement distance for a respective tooth of the plurality of teeth from the initial position to the final position. In some embodiments, the staging processing engine 212 may identify the maximum movement distance by comparing the movement distances for each of the plurality of teeth. The staging processing engine 212 may determine the movement distances as a straight line path from a point on the teeth from their initial position to the final position. In some embodiments, the staging processing engine 212 may determine the movement distances as a length of the path in which the respective teeth travel from the initial position to the final position. The staging processing engine 212 may identify the maximum movement distance based on which of the teeth have the greatest movement distance.

At step 1604, and optionally, the staging processing engine 212 determines a number of stages. In some embodiments, the staging processing engine 212 may determine a number of stages to generate based on the maximum movement distance. The staging processing engine 212 may determine the number of stages based on the maximum movement distance and a single stage movement limit. The staging processing engine 212 may determine the number of stages by dividing the maximum movement distance and single stage movement limit, and rounding up the resulting number.

At step 1606, the staging processing engine 212 generates a movement vector. In some embodiments, the staging processing engine 212 may generate a first movement vector for a first tooth of the plurality of teeth for a first intermediate 3D representation. The movement vector may include a first movement direction from the initial position for the first tooth towards the final position of the first tooth, and a first movement magnitude corresponding to a distance between the initial position and the final position. The staging processing engine 212 may generate the movement vectors based on the path for the teeth from the initial position to the final position. The staging processing engine 212 may generate the movement vectors to have a direction component which generally follows the path, and a magnitude which corresponds to the movement distance for the tooth.

In some embodiments, the first movement magnitude is a function of the movement distance between the initial position and the final position and the determined number of stages. For example, the movement magnitudes may be equal to the movement distance divided by the determined number of stages. In some embodiments, the first movement magnitude is equal to a single stage movement limit. The staging processing engine 212 may compute the movement magnitude as the single stage movement limit, unless the movement distance remaining along the path is less than the single stage movement limit. As such, the staging processing engine 212 may determine movement magnitudes for a given tooth that are equal to the single stage movement limit, until the remaining distance for the path of the tooth is less than the movement limit.

At step 1608, the staging processing engine 212 determines whether a collision is detected. In some embodiments, the staging processing engine 212 may detect a collision between the first tooth and a second tooth of the plurality of teeth based on the movement vector for the first tooth. For example, the staging processing engine 212 may project the each of the teeth in a 3D representation (e.g., to generate a subsequent 3D representation) based on the movement vector. The staging processing engine 212 may project each of the teeth in the direction according to the direction component and a length along the path based on the magnitude component of the movement vector. The staging processing engine 212 may detect the collision based on two or more teeth overlapping one another following projection. In some embodiments, the staging processing engine 212 may determine an intrusion depth of the collision between the first tooth and the second tooth. The staging processing engine 212 may determine the intrusion depth based on a distance between outermost points on the teeth at the overlapping portion of the teeth.

At step 1610, if there is a collision detected at step 1608, the staging processing engine 212 may determine whether the collision is a multi-tooth collision. In some embodiments, the staging processing engine 212 may detect the collision between the first tooth, the second tooth, and a third tooth of the plurality of teeth based on the movement vector for the first tooth. Where, at step 1610, the staging processing engine 212 determines the collision is a multi-tooth collision, at step 1612, the staging processing engine 212 may identify movement vectors for adjacent teeth. On the other hand, where the staging processing engine 212 determines that the collision is a collision between two teeth, the method 1600 may proceed to step 1614.

At step 1614, the staging processing engine 212 generates a corrective (or second) movement vector. In some embodiments, the staging processing engine 212 generates a second movement vector for the first tooth for the first intermediate 3D representation. In some embodiments, the second movement vector may have the first movement direction and a second movement magnitude. In other words, the second movement vector may have the same movement direction as the first movement vector generated at step 1606. However, the second movement vector may have a magnitude with is different form the movement vector generated at step 1606. In some embodiments, the second magnitude is less than the first magnitude. In some embodiments, the second movement vector may have a different movement direction than the movement direction of the first movement vector generated at step 1606. For example, the first movement direction may be along (e.g., on) the path from the initial position of the tooth and a final position, and the second movement direction may be adjacent to the path. In this regard, both the first and second movement directions may be towards the final position, with the first movement direction being on the path towards the final position and the second movement direction being adjacent to the path towards the final position.

In some embodiments, the second magnitude and/or the second direction is determined based on the intrusion depth of the first tooth and the second tooth. For example, the staging processing engine 212 may determine the second magnitude by subtracting the intrusion depth (or a portion of the intrusion depth) from the magnitude of the first movement vector generated at step 1606. As another example, the staging processing engine 212 may determine the second direction by rotating the second direction from the path at an angle which corresponds to the intrusion depth.

In some embodiments, the staging processing engine 212 may generate the second movement vector for the first tooth based on the first movement vector for the first tooth and a third movement vector for the third tooth (e.g., where the staging processing engine 212 determines that the first tooth is colliding with a second and a third tooth). In some embodiments, the second movement vector is based on a sum of the first movement vector and the third movement vector. The second movement vector may be the sum responsive to detecting the collision between the first tooth, the second tooth, and the third tooth.

Following generating the corrective movement vector at step 1614, the method 1600 may return to step 1606, where the staging processing engine 212 determines whether a collision is detected. As such, the staging processing engine 212 may iteratively loop between steps 1608 through step 1614 until the staging processing engine 212 does not detect a collision. The staging processing engine 212 may repeat this process until each of stages (e.g., determined to generate at step 1604) are generated by the staging processing engine 212, and no collisions are detected by the staging processing engine 212. The staging processing engine 212 may generate the stages based on the projections of the teeth according to the initial (or corrective) movement vectors for the teeth.

In instances where the staging processing engine 212 generates a corrective movement vector which includes a direction component that moves the tooth off the path to the final position, the staging processing engine 212 may generate one or more subsequent movement vectors which bring the tooth back on the path to the final position. The subsequent movement vector(s) may be for subsequent stages of the treatment plan. In some instances, the subsequent movement vector(s) may be towards the final position such that the tooth is moved along the path (and on the path) at a position which is closer than a position of the tooth. In some embodiments, at each stage of the treatment plan, the teeth may move towards the final position (e.g., either on the path or, in the event of a detected collision, adjacent to the path). In this regard, rather than round-tripping a tooth, the tooth may be moving towards the final position.

At step 1616, the staging processing engine 212 manufactures a plurality of dental aligners. Step 1616 may be substantially the same as step 1508 described above with reference to FIG. 15.

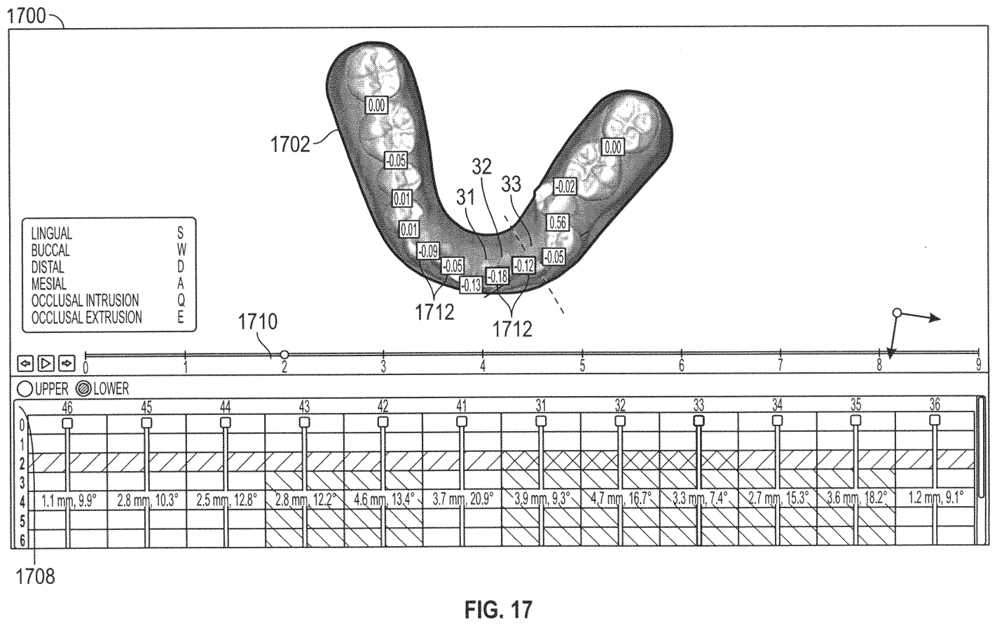

Referring now to FIG. 17, depicted is a user interface 1700 showing a 3D model 1702 of a dentition, according to an illustrative embodiment. The user interface 1700 may be displayed or otherwise rendered on a treatment planning terminal 108 described above. The user interface 1700 may include regions for selecting various steps of generating the treatment plan as described above (e.g., segmentation by the segmentation processing engine 206, matching by the geometry processing engine 208, final position by the final position processing engine 210, etc.). Upon selecting a staging button, the user interface 1700 may be rendered on the treatment planning terminal 108 and used to generate stages for the treatment plan as described herein. In some embodiments, the treatment planning system 102 may generate default stages for the treatment plan, which may include moving each of the teeth along the path a predetermined or preset amount towards the final position. However, the default stages may result in one or more collisions.

The user interface 1700 is shown to include a staging region 1708 which shows movement of each of the individual teeth in the 3D model 1702. The teeth may be represented in the staging region 1708 according to the teeth numbers described above with reference to the segmentation processing engine 206. The staging region 1708 may include rows which represent movement at each stage, and columns which represent each of the teeth. Where a collision is detected between two or more teeth in any stage of the default staging, the staging region 1708 may include a highlighting or fill which identifies the collision, the tooth, and the stage. For example, and as shown in FIG. 17, at the second stage (shown as row 2), a collision may be detected between teeth numbers 31 and 32, and between teeth numbers 32 and 33. As such, the staging region 1708 may provide a visual identification of any collisions detected, which indicates which teeth are detected as colliding and in which stage the collision is detected.

The user interface 1700 may include a slide bar 1710 which is configured to receive a selection of a particular stage of the treatment plan. A user may select a play button to show a visual progression of the teeth from the initial position (e.g., at stage 0) to the final position (e.g., at stage 9 in the example shown in FIG. 17). In the example shown in FIG. 17, a user may have selected stage 2 on the slide bar 1710. Selecting a particular stage on the slide bar 1710 may highlight the corresponding row in the staging region 1708 of the user interface.

The user interface 1700 is shown include interproximal overlays 1712 which show an interproximal space (e.g., a measure of the space between two teeth) or an intrusion depth in the event of a collision. the corresponding interproximal overlay 1712 may be bound in a different color to provide visual feedback of the collision. In the example shown in FIG. 17, where the collision is detected between tooth numbers 31 and 32, and tooth numbers 32 and 33, the interproximal overlays 1712 between those respective teeth may be bound and show the intrusion depth of the collision between the respective teeth. In some embodiments, where a collision is detected between two teeth, the 3D model 1700 may be updated to modify a shading between the two teeth. For example, the shading between the two teeth may be red to identify the collision. However, where a collision is not detected, the shading may be green. Such feedback may be in addition to or supplement the interproximal overlays 1712, according to various embodiments.

In some instances, a user may manually move the teeth to avoid a collision. For example, a user may select a particular tooth on the 3D model 1702 (such as tooth 33 in the example shown in FIG. 17) and provide keyed inputs (such as those shown in the table above the slider bar 1710), or slide the tooth along axes in the buccal-lingual or mesial-distal direction. However, this process can be time consuming, and may result in inconsistencies in treatment planning across different users.

In some embodiments, the user interface 1700 may include an optimize stages button or other user interface element. Upon selecting the optimize stages button, the staging processing engine may execute the methods described herein to automatically generate stages for the treatment plan which avoid the collisions between the teeth that may result from the default staging described above.

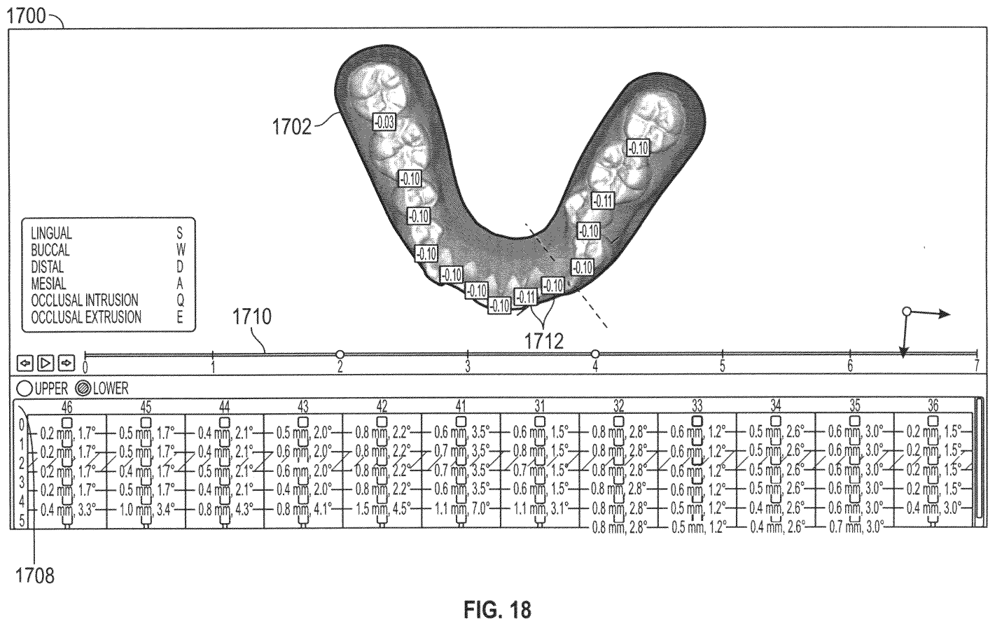

Referring now to FIG. 18, depicted is the user interface 1700 following generating stages of a treatment plan, according to an illustrative embodiment. The user interface 1700 may be rendered responsive to selecting an optimize stages button or user interface element, which may be included on the user interface 1700 of FIG. 17. The staging processing engine 212 may be configured to execute one or more of the methods described herein to automatically generate the stages of the treatment plan responsive to selecting the optimized stages button. As shown in FIG. 18, the staging region 1708 does not include any highlighting or filling which indicates collisions between two or more teeth at any stage of the treatment plan. Continuing the example shown in FIG. 17, at the second stage, the previously-identified collision between teeth numbers 31 and 32, and teeth numbers 32 and 33 is no longer detected and, as such, the corresponding boxes in the staging region 1708 is not highlighted or filled, and the interproximal overlays 1712 are not bound.

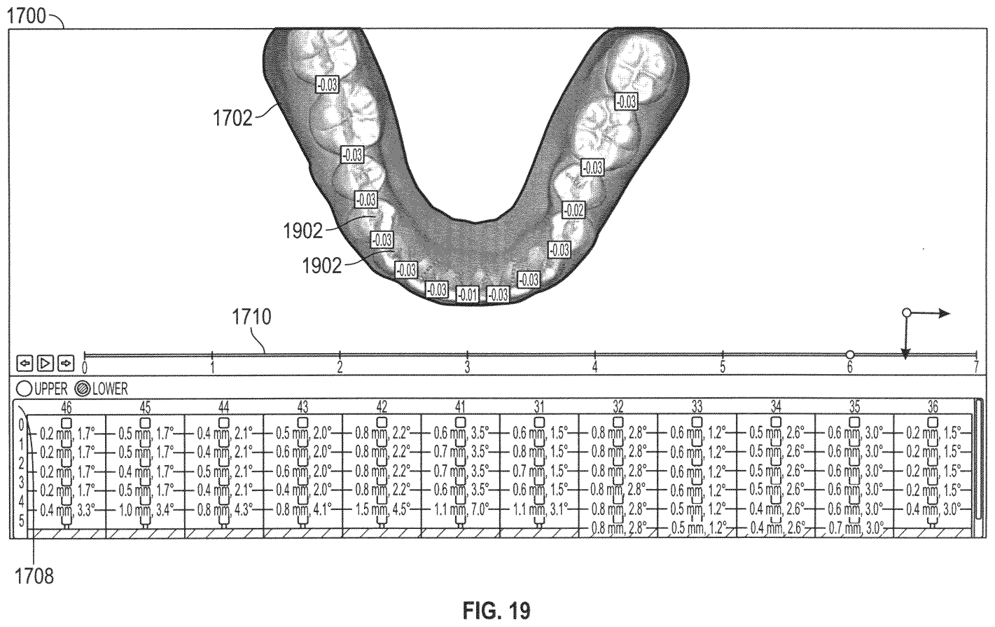

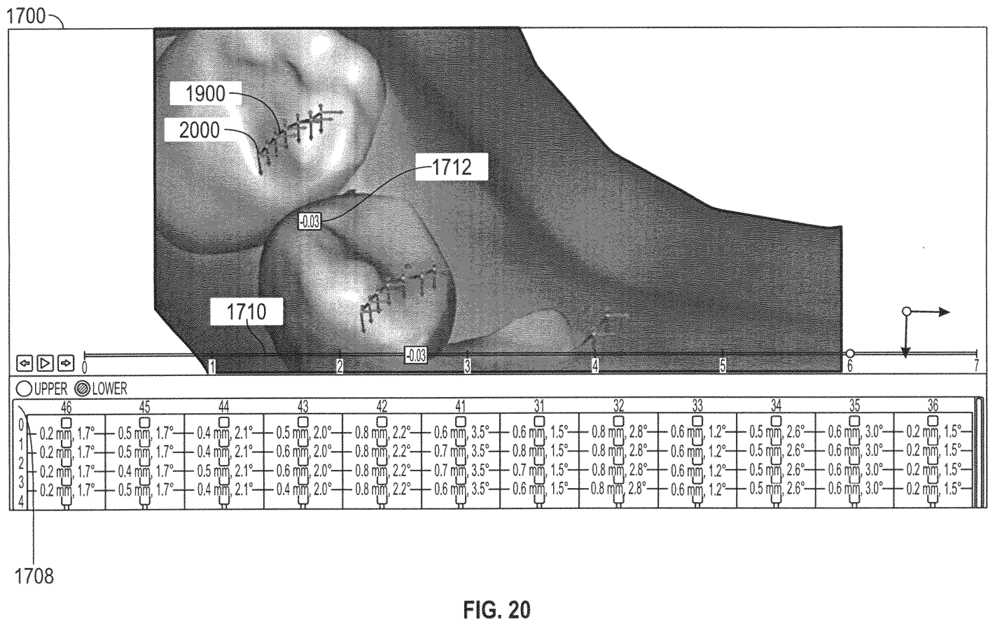

Referring now to FIG. 19 and FIG. 20, depicted is the user interface 1700 including views of the trajectory of the teeth, according to an illustrative embodiment. In some embodiments, the user interface 1700 may include a “show teeth trajectory” selectable field. Upon selecting the field, a trajectory or path 1902 of each of the teeth may be overlaid onto the respective teeth. The path 1902 may be similar to the path described above with reference to FIG. 10-FIG. 14. The path 1902 may include points 2000 showing a position of the teeth (e.g., a center of the teeth) at each stage as the teeth move from the initial position to the final position. As such, each point 2000 may be representative of a respective stage of the treatment plan. Where a user zooms in on a particular path (as shown in FIG. 20), the points 2000 may include the movement vectors described above with reference to FIG. 10-FIG. 14.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (e.g., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the F. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the systems, apparatuses, and methods shown in the various exemplary embodiments is illustrative only. Additionally, any element disclosed in one embodiment may be incorporated or utilized with any other embodiment disclosed herein. For example, any of the exemplary embodiments described in this application can be incorporated with any of the other exemplary embodiment described in the application. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A method, comprising:
receiving, by one or more processors, a first three-dimensional (3D) representation of a dentition including representations of a plurality of teeth of the dentition in an initial position;
determining, by the one or more processors, a second 3D representation including representations of the plurality of teeth in a final position; and
generating, by the one or more processors, one or more stages including intermediate 3D representations of the dentition, the intermediate 3D representations including representations of at least some of the plurality of teeth progressing from the initial position to the final position, wherein generating the one or more stages comprises:
generating, by the one or more processors, a first movement vector for a first tooth of the plurality of teeth for a first intermediate 3D representation, the first movement vector including a first movement direction from the initial position for the first tooth towards the final position of the first tooth, and a first movement magnitude corresponding to a distance between the initial position and the final position;
detecting, by the one or more processors, a collision between the first tooth and a second tooth of the plurality of teeth based on the first movement vector for the first tooth;
generating, by the one or more processors, a second movement vector for the first tooth for the first intermediate 3D representation, the second movement vector having at least one of a second movement direction towards the final position or a second movement magnitude; and
generating, by the one or more processors, a first stage of the one or more stages according to the second movement vector for the first tooth.

2. The method of claim 1, wherein the second movement vector has the second movement direction towards the final position, and wherein the first tooth is moved in the first stage from a first position to a second position adjacent to a path from the initial position to the final position, and wherein the method further comprises:
generating, by the one or more processors, for a second intermediate 3D representation for a second stage subsequent to the first stage, a third movement vector for the first tooth, the third movement vector including a third movement direction from the second position for the first tooth towards the final position of the first tooth, and the first movement magnitude; and
generating, by the one or more processors, a second stage of the one or more stages according to the third movement vector for the first tooth, wherein the first tooth is moved in the second stage from the second position adjacent to the path to a third position on the path from the initial position to the final position.

3. The method of claim 2, wherein the second position is closer to the final position than the first position, and wherein the third position is closer to the final position than the second position.

4. The method of claim 1, wherein the first movement magnitude is a function of the distance between the initial position and the final position and the determined number of stages.

5. The method of claim 1, wherein the first movement magnitude is equal to a single stage movement limit.

6. The method of claim 1, further comprising determining an intrusion depth of the collision between the first tooth and the second tooth,
wherein the second movement vector is iteratively determined based on the intrusion depth of the first tooth and the second tooth, wherein the second movement direction or second movement magnitude are modified at each iteration based on the intrusion depth.

7. The method of claim 1, wherein detecting the collision comprises detecting the collision between the first tooth, the second tooth, and a third tooth of the plurality of teeth based on the movement vector for the first tooth; and
wherein generating the second movement vector for the first tooth comprises generating the second movement vector for the first tooth based on the first movement vector for the first tooth and a third movement vector for the third tooth.

8. The method of claim 7, wherein the second movement direction is a sum of the first movement direction of the first movement vector and a third movement direction of the third movement vector responsive to detecting the collision between the first tooth, the second tooth, and the third tooth.

9. The method of claim 1, wherein each of the at least some of the plurality of teeth being moved from the initial position to the final position are moved at least some distance for each of the one or more stages, and wherein the at least some distance is along a path from the initial position to the final position.

10. The method of claim 9, wherein the path for the first tooth includes translational and rotational movements, and wherein the first and second movement vectors have a common translational movement.

11. The method of claim 1, wherein the second magnitude is less than the first magnitude.

12. The method of claim 1, further comprising manufacturing, for each stage of the one or more stages, a plurality of aligners configured to move the at least some teeth from the initial position to the final position.

13. The method of claim 1, further comprising:
identifying, by the one or more processors, for the plurality of teeth, a maximum movement distance for a respective tooth of the plurality of teeth from the initial position to the final position; and
determining, by the one or more processors, a number of stages to generate based on the maximum movement distance.

14. A treatment planning system, comprising:
one or more processors; and
memory storing instructions that, when executed by the one or more processors, cause the one or more processors to:
receive a first three-dimensional (3D) representation of a dentition including representations of a plurality of teeth of the dentition in an initial position;
determine a second 3D representation including representations of the plurality of teeth in a final position; and
generate one or more stages including intermediate 3D representations of the dentition, the intermediate 3D representations including representations of at least some of the plurality of teeth progressing from the initial position to the final position, wherein generating the one or more stages comprises:
generating a first movement vector for a first tooth of the plurality of teeth for a first intermediate 3D representation, the first movement vector including a first movement direction from the initial position for the first tooth towards the final position of the first tooth, and a first movement magnitude corresponding to a distance between the initial position and the final position;
detecting a collision between the first tooth and a second tooth of the plurality of teeth based on the first movement vector for the first tooth;
generating a second movement vector for the first tooth for the first intermediate 3D representation, the second movement vector having at least one of a second movement direction towards the final position or a second movement magnitude; and
generating a first stage of the one or more stages according to the second movement vector for the first tooth.

15. The treatment planning system of claim 14, wherein the first movement magnitude is at least one of a function of the distance between the initial position and the final position and a determined number of stages, or equal to a single stage movement limit.

16. The treatment planning system of claim 14, wherein the instructions further cause the one or more processors to determine an intrusion depth of the collision between the first tooth and the second tooth,
wherein the second movement vector is iteratively determined based on the intrusion depth of the first tooth and the second tooth.

17. The treatment planning system of claim 14, wherein detecting the collision comprises detecting the collision between the first tooth, the second tooth, and a third tooth of the plurality of teeth based on the movement vector for the first tooth; and
wherein generating the second movement vector for the first tooth comprises generating the second movement vector for the first tooth based on the first movement vector for the first tooth and a third movement vector for the third tooth.

18. The treatment planning system of claim 17, wherein the second movement direction is a sum of the first movement direction of the first movement vector and a third movement direction of the third movement vector responsive to detecting the collision between the first tooth, the second tooth, and the third tooth.

19. The treatment planning system of claim 14, wherein each of the at least some of the plurality of teeth being moved from the initial position to the final position are moved at least some distance for each of the one or more stages, and wherein the at least some distance is along a path from the initial position to the final position.

20. The treatment planning system of claim 19, wherein the path for the first tooth includes translational and rotational movements, and wherein the first and second movement vectors have a common translational movement.

21. The treatment planning system of claim 14, wherein the second magnitude is less than the first magnitude.

22. The treatment planning system of claim 14, wherein the second movement vector has the second movement direction towards the final position, and wherein the first tooth is moved in the first stage from a first position to a second position adjacent to a path from the initial position to the final position, and wherein generating the one or more stages further comprises:
generating, for a second intermediate 3D representation for a second stage subsequent to the first stage, a third movement vector for the first tooth, the third movement vector including a third movement direction from the second position for the first tooth towards the final position of the first tooth, and the first movement magnitude; and
generating, a second stage of the one or more stages according to the third movement vector for the first tooth, wherein the first tooth is moved in the second stage from the second position adjacent to the path to a third position on the path from the initial position to the final position.

23. The treatment planning system of claim 22, wherein the second position is closer to the final position than the first position, and wherein the third position is closer to the final position than the second position.

24. A non-transitory computer readable medium storing instructions that, when executed by one or more processors, cause the one or more processors to:
receive a first three-dimensional (3D) representation of a dentition including representations of a plurality of teeth of the dentition in an initial position;
determine a second 3D representation including representations of the plurality of teeth in a final position; and
generate one or more stages including intermediate 3D representations of the dentition, the intermediate 3D representations including representations of at least some of the plurality of teeth progressing from the initial position to the final position, wherein generating the one or more stages comprises:
identifying, for the plurality of teeth, a maximum movement distance for a respective tooth of the plurality of teeth from the initial position to the final position;
determining a number of stages to generate based on the maximum movement distance;
generating a first movement vector for a first tooth of the plurality of teeth for a first intermediate 3D representation, the first movement vector including a first movement direction from the initial position for the first tooth towards the final position of the first tooth, and a first movement magnitude corresponding to a distance between the initial position and the final position;
detecting a collision between the first tooth and a second tooth of the plurality of teeth based on the first movement vector for the first tooth;
generating a second movement vector for the first tooth for the first intermediate 3D representation, the second movement vector having the first movement direction and a second movement magnitude; and
generating a first stage of the one or more stages according to the second movement vector for the first tooth.
